(19) United States
(12) Patent Application Publication
(10) Pub. No.: US 2026/0191705 A1
(43) Pub. Date: Jul. 9, 2026

(54) ABSORBENT ARTICLE WITH RISE ADJUSTMENT FEATURE FOR BETTER FIT AND COMFORT

(71) Applicant: Kimberly-Clark Worldwide, Inc., Neenah, WI (US)

(72) Inventors: Juliana Rodrigues Calil, São Paulo (BR); Stephanie Anne-Genevieve Riemer, Hortonville, WI (US); Regina Helen Herrmann Chalmers, Santana De Parnaiba (BR)

(21) Appl. No.: 19/132,635

(22) PCT Filed: Dec. 2, 2022
(86) PCT No.: PCT/US2022/080809

§ 371 Date: May 23, 2025

(51) Int. Cl. A61F 13/56; A61F 13/49; A61F 13/493; A61F 13/62

(52) U.S. Cl. CPC A61F 13/5638; A61F 13/49004; A61F 13/493; A61F 13/62

(57) ABSTRACT

An absorbent article includes a chassis having an outer cover and a rise adjustment feature for adjusting a rise of the absorbent article. The rise adjustment feature may be accessible from an interior of the absorbent article. The rise adjustment feature includes at least one first fastening element on a connecting member, such as a strap, and a second fastening element on the outer cover. The rise of the absorbent article may be adjusted by engaging the at least one first fastening element with the second fastening element.

### FIELD OF THE INVENTION

The present disclosure relates generally to absorbent articles intended for personal wear, and more particularly to reusable absorbent articles with rise adjustment features for better fit and comfort to the wearer.

### BACKGROUND

Many absorbent articles intended for personal wear, such as diapers, training pants, feminine hygiene products, adult incontinence products, bandages, medical garments, and the like are designed to be sufficiently absorbent to absorb moisture from liquid body exudates including urine, menses, blood, etc., away from the wearer to reduce skin irritation caused by prolonged wetness exposure. Diapers, as an example, are typically placed and secured on a wearer using a set of primary fastening tabs, such as adhesive tabs or mechanical (e.g., hook or loop) fastening system tabs, and left in place to absorb solid, liquid, and semi-liquid waste.

Diapers are usually characterized as disposable or reusable. Disposable diapers have dominated the market for decades and are commonly provided in customized sizes to fit children in various stages of growth based on weight and height. Once a child outgrows one size diaper, the next size range of diapers must be bought.

In contrast to the single-use nature of disposable diapers, reusable diapers may be wholly or partially reusable such that the general construction can be used repeatedly and often are intended to fit children in different stages of growth. Reusable cloth diapers existed long before disposable diapers, but they were generally ill fitting, bulky, and often failed to effectively contain liquid and other waste. Improvements have been made to reusable diapers to address the drawbacks of traditional cloth diapers. For example, many reusable diapers include a durable outer shell in which is positioned a disposable or reusable absorbent component that may be removed and replaced. Even with such improvements, existing reusable diapers still have issues with fit and comfort compared to disposable diapers, and further improvements are needed to allow reusable diapers to accommodate a child, particularly as the child grows.

### SUMMARY

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various embodiments of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

According to certain embodiments, an absorbent article includes a rise adjustment feature. The rise adjustment feature includes a strap with at least one first fastening element, and the strap may be connectable to a body-facing side of an outer cover of the absorbent article. The rise adjustment feature also includes a second fastening element connected to the outer cover. A rise of the absorbent article may be adjusted by engaging the at least one first fastening element with the second fastening element.

According to certain embodiments, an absorbent article includes a rise adjustment feature that is accessible from an interior of the absorbent article. The rise adjustment feature includes at least one first fastening element and a second fastening element. The at least one first fastening element is coupled to a connecting member, and the at least one first fastening element is selectively engageable with the second fastening element to adjust a rise of the absorbent article.

According to certain embodiments, an absorbent article includes an outer cover and a rise adjustment feature. The outer cover includes a body-facing side and an outward-facing side. The rise adjustment feature includes connecting member, at least one first fastening element, and a second fastening element. The connecting member is connected to the outer cover and extends over a portion of the body-facing side. The at least one first fastening element is coupled to the connecting member. In some embodiments, the at least one first fastening element is movable relative to the outer cover. The second fastening element is connected to the outer cover. A rise of the absorbent article is adjusted by engaging the at least one first fastening element with the second fastening element.

Various implementations described in the present disclosure can include additional systems, methods, features, and advantages, which cannot necessarily be expressly disclosed herein but will be apparent to one of ordinary skill in the art upon examination of the following, detailed description and accompanying drawings. It is intended that all such systems, methods, features, and advantages be included within the present disclosure and protected by the accompanying claims.

### BRIEF DESCRIPTION OF THE DRAWINGS

The features and components of the following figures are illustrated to emphasize the general principles of the present disclosure. Corresponding features and components throughout the figures can be designated by matching reference characters for the sake of consistency and clarity.

FIG. 1 illustrates a diaper on a user according to embodiments.

FIG. 2 is a top plan view of the diaper of FIG. 1 in an unfolded configuration and showing a side of the diaper which faces away from the wearer when the diaper is worn.

FIG. 3 is a bottom plan view of the diaper of FIG. 1 in the unfolded configuration and showing a body-side of the diaper which faces the wearer when the diaper is worn along with an absorbent component.

FIG. 4 is another bottom plan view of the diaper of FIG. 1 with the absorbent component removed.

FIG. 5 is another bottom plan view of the diaper of FIG. 1 with a liner material removed.

FIG. 6 is another bottom plan view of the diaper of FIG. 1 with first fastening elements of a rise adjustment feature removed.

FIG. 7 illustrates first fastening elements of the rise adjustment feature of the diaper of FIG. 1 according to embodiments.

FIG. 8 is a perspective view of a portion of the diaper of FIG. 1.

FIG. 9 is another perspective view of a portion of the diaper of FIG. 1.

### DETAILED DESCRIPTION

Described herein are rise adjustment features for absorbent articles, and absorbent articles incorporating such rise adjustment features. In certain embodiments, the rise adjustment features described herein may provide better fit and comfort for a wearer while allowing for the overall rise or length of the absorbent article to be adjusted. In various embodiments, the rise adjustment features described herein are accessible from an interior of the absorbent article, which may provide a more durable absorbent article compared to traditional approaches. In some embodiments, the rise adjustment features described herein are enclosed within the interior of the absorbent article to prevent and/or minimize contact with the skin of the wearer. In certain aspects, enclosing the rise adjustment features may further improve durability of the absorbent article. Enclosing the rise adjustment feature may also minimize or prevent the wearer from accessing the rise adjustment feature during usage, which may provide a safety advantage for certain wearers (e.g., infants). Various other benefits and advantages may be realized with the systems and methods provided herein, and the aforementioned advantages should not be considered limiting.

While the rise adjustment features described below are discussed in the context of diapers as absorbent articles, and specifically reusable diapers, they are by no means so limited. Rather, embodiments of the rise adjustment features described herein may be used for rise adjustment of various absorbent articles as desired.

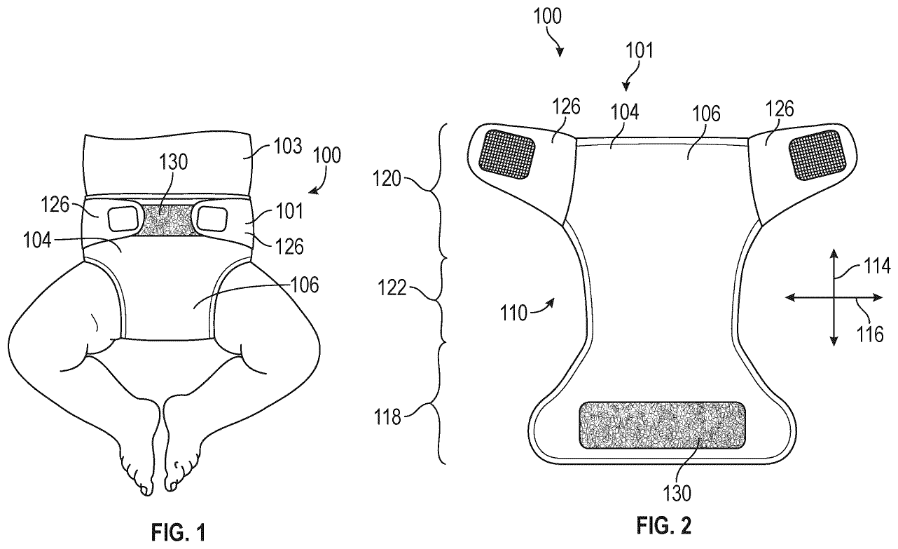

FIGS. 1-9 illustrate an example of an absorbent article 100 with a rise adjustment feature 102 according to embodiments. In FIGS. 1-9, the absorbent article 100 is a reusable diaper 101, however, in other embodiments, other absorbent articles may incorporate the rise adjustment feature 102, and/or the diaper need not be a reusable diaper 101.

Referring to FIGS. 2-4, the diaper 101 generally includes a chassis 104 having an outer cover 106 and a liner material 108. In some embodiments, the outer cover 106 is substantially liquid-impervious to thereby prevent moisture from penetrating through the chassis 104. Suitable materials include, but are not limited to, waterproof materials, water resistant materials, coated materials, laminated materials, nonwovens, etc. Polymeric materials such as but not limited to polyester and polyurethane may be particularly suitable. The liner material 108 is located more proximate to, or directly in contact with, the wearer and thus may be formed of materials or in a way that imparts comfort but yet is still durable. Such materials include, but are not limited to, polymeric materials (e.g., polyester, polyurethane, etc.) and natural and synthetic cellulosics (e.g., cotton, rayon, acetate, triacetate, and lyocell fibers). In some embodiments, the liner material 108 is a fleece or microfleece material.

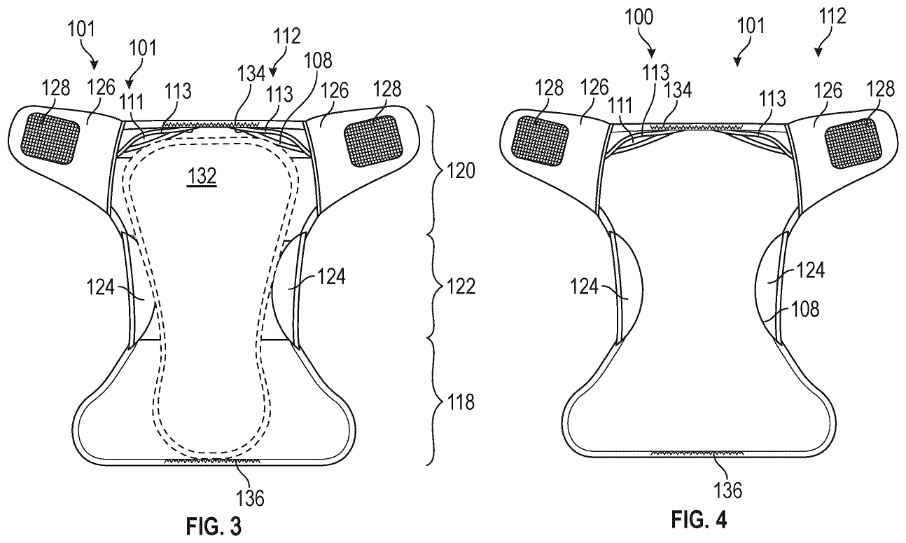

FIG. 2 illustrates the diaper 101 in an unfolded and laid flat condition to show the outward-facing side 110 of the chassis 104, and FIGS. 3 and 4 illustrate the diaper 101 in the unfolded and laid flat condition to show the body-facing side 112 of the chassis 104. FIGS. 2-6 illustrate the diaper 101 in an unstretched configuration (e.g., not stretched in a lateral direction 116). When worn and/or when stretched in the lateral direction 116, back ears 126 may generally be in line with the back portion 120 and not at an angle as illustrated in FIGS. 2-6.

The diaper 101 generally has a longitudinal direction 114 and the lateral direction 116. In the longitudinal direction 114, the diaper 101 includes a front portion 118, a back portion 120, and a crotch portion 122 between the front portion 118 and the back portion 120. In some embodiments, the diaper 101 may include a front waist elastic 136 and/or a back waist elastic 134. In the illustrated embodiment, for example, the diaper 101 has a back waist elastic 134 and a front waist elastic 136. The back waist elastic 134 and the front waist elastic 136 may be arranged to draw and hold the diaper 101 against the wearer, particularly against the waist of the wearer. As used herein, “elastic,” “elastomeric,” and the like refer to the ability of a material or composite to be elongated by at least about 50 percent and upon relaxation to return to within at least 50 percent of its original length.

The back portion 120 of the diaper 101 includes a pair of back ears 126. Each back ear 126 may include a first main fastening component 128 as part of a fastening system used to secure the diaper 101 around the waist of the wearer. The front portion 118 of the diaper 101 may include second main fastening component 130, and the first main fastening components 128 may engage the second main fastening component when the diaper 101 is moved from the unfastened configuration to a wear configuration and to secure the diaper 101 around the waist of the wearer. FIG. 1 illustrates the diaper 101 in the wear configuration on the wearer 103 and with the first main fastening components 128 engaged with the second main fastening component 130. The first and second main fastening components 128, 130 may be various suitable devices and mechanisms suitable for allowing adjustment of the diaper 101 from the unfastened configuration to the wear configuration and vice versa. As non-limiting examples, the first and second main fastening components 128, 130 may be hook and loop fasteners, snaps, clips, clasps, adhesive fasteners, cohesive fasteners, mechanical fasteners, male/female mating components, combinations thereof, and/or other suitable features as desired.

Leg cuffs 124 may extend in the longitudinal direction 114 and may be adapted to fit about legs of a wearer in use and to serve as a mechanical barrier to the lateral flow of body exudates. In some embodiments, the leg cuffs 124 may be portions of the outer cover 106. However, in other embodiments, the leg cuffs 124 may be separate materials (e.g., strands of leg elastics) that are joined to the chassis 104.

An absorbent core or material 132 may be provided with the diaper 101. The absorbent material 132 is generally conformable and capable of absorbing and retaining liquid body exudates. The absorbent material 132 can include superabsorbent material, staple fibers, binder, and the like, and combinations thereof as is known in the art. The absorbent material 132 may have any of a number of shapes and sizes. For example, the absorbent material 132 may be formed of any suitable material or assembly of materials, including, but not limited to, those disclosed in U.S. Pat. No. 9,011,403, issued Apr. 21, 2015 and entitled “Absorbent Insert”; U.S. Pat. No. 6,710,225, issued Mar. 23, 2004 and entitled “Layered Absorbent Structure with a Zoned Basis Weight”; U.S. application Ser. No. 16/086,033, published as US 2020/0289342 on Sep. 17, 2020 and entitled “Absorbent Cores and Methods for Forming Absorbent Cores”, and PCT/US2020/047736, published as WO 2022/046030 on Mar. 3, 2022 and entitled “Absorbent Structures and Methods for Manufacturing Absorbent Structures,” the entirety of each of which is herein incorporated by reference. In the context of reusable diapers, the absorbent material 132 may be a disposable insert that is removed after use and replaced within the chassis 104 by a new disposable insert, a reusable insert that is cleaned after use and then replaced within the chassis 104, and/or permanently incorporated on or into the chassis 104 such that both the chassis 104 with associated absorbent material 132 are cleaned and reused after use. The absorbent material 132 may be retained within the chassis 104 in myriad ways, such as via insertion into a pocket within the chassis 104 or via mechanical or chemical retention features (e.g., hook and loop fasteners, snaps, adhesives, etc.).

The liner material 108 may be attached to the outer cover 106 and covers at least a portion of the body-facing side 112. In certain embodiments, the liner material 108 is attached to the outer cover 106 such that a pocket 111 is defined between the liner material 108 and the outer cover 106. In various embodiments, and as illustrated in FIG. 4, the liner material 108 is attached to the outer cover 106 at various locations such that one or more openings 113 to the pocket Ill are defined.

In the embodiment illustrated, the diaper 101 is a reusable diaper 101, and the absorbent material 132 may be selectively removed and replaced as needed. In other words, a used absorbent material 132 may be replaced with an unused absorbent material 132 while utilizing the same chassis 104. The absorbent material 132 itself may be disposable or reusable as desired. In other embodiments, the diaper 101 need not be reusable (e.g., it may be a disposable diaper), and in such embodiments, the absorbent material 132 may be positioned within the chassis 104 as desired.

Padding (not illustrated) may optionally be provided with the diaper 101. When included, the padding may be provided in various locations, and the padding may be in various shapes or sizes as desired. In one non-limiting examples, padding may be provided between the liner material 108 and the outer cover 106. The padding may be permanently retained in position or may be removable from the chassis 104 as desired.

The absorbent article 100 includes at least one rise adjustment feature 102 such that a rise of the absorbent article 100 (or length in the longitudinal direction 114) may be adjusted as desired. In the embodiment illustrated in FIGS. 1-9, the absorbent article includes two rise adjustment features 102; however, the number of rise adjustment features 102 should not be considered limiting. In certain embodiments, and as best illustrated in FIGS. 4-9, the rise adjustment features 102 may be provided adjacent the back portion 120 of the diaper 101. However, in other embodiments, the rise adjustment features 102 may be provided in other locations on the diaper 101, such as but not limited to the front portion 118.

In some embodiments, the rise adjustment feature 102 may be provided within the pocket 111 and between the outer cover 106 and the liner material 108 so as to be generally hidden from view from the outward-facing side 110 and the body-facing side 112 of the chassis 104. Such positioning may impart a more polished appearance to the diaper 101, may minimize and/or prevent contact between the rise adjustment feature 102 and the wearer 103, and/or may protect and provide improved durability to the rise adjustment feature 102.

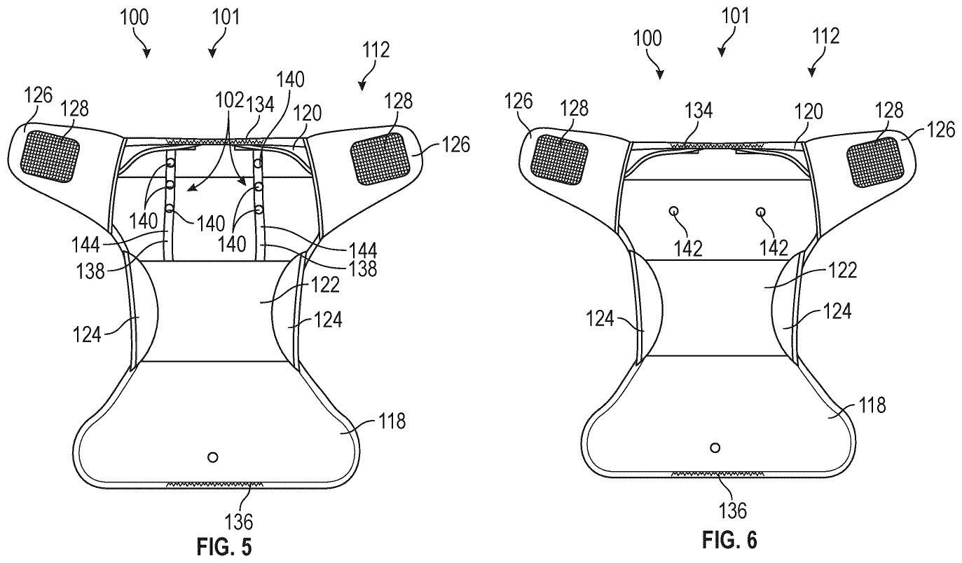

Referring to FIGS. 5-9, each rise adjustment feature 102 includes a connecting member 138, at least one first fastening element 140 and a second fastening element 142.

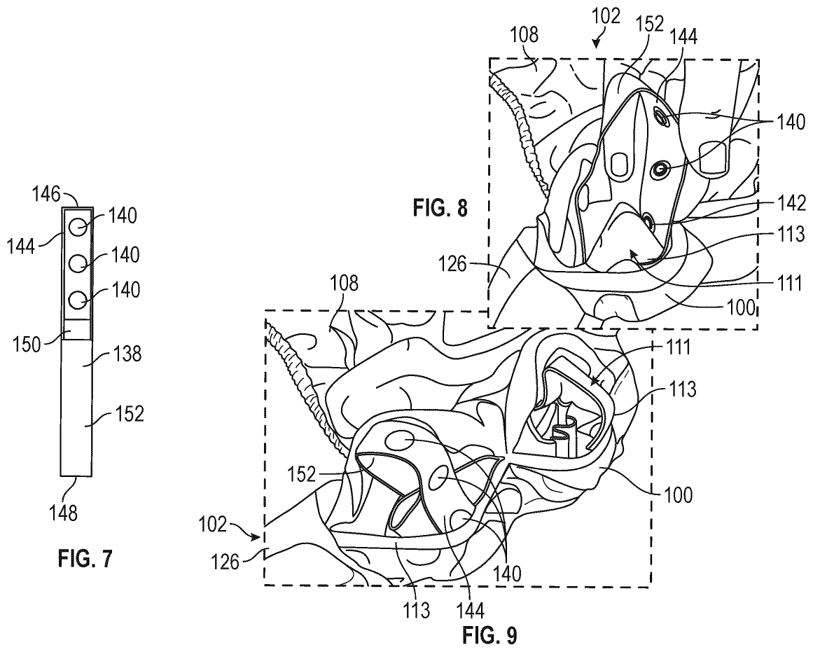

The connecting member 138 is connected to the outer cover 106 using various techniques or mechanisms as desired. In certain embodiments, and as illustrated in FIG. 7, for example, the connecting member 138 may be a strap 144 having opposing ends 146, 148 and a body portion 150 between the ends 146, 148. In other embodiments, the strap 144 and/or the connecting member 138 may have other shapes or profiles as desired. In various embodiments, at least a portion 152 of the strap 144 may be an elastic material, and optionally the entire strap 144 may be elastic material. In certain embodiments, the connecting member 138 and/or the strap 144 that is at least partially elastic may provide improved comfort and fit to the wearer 103, particularly if the rise of the diaper 101 is adjusted. In other embodiments, the strap 144 need not be elastic. In certain embodiments, at least one of the ends 146, 148 may be connected to the outer cover 106, and in some embodiments, both ends 146, 148 may be connected to the outer cover 106. In certain aspects, the body portion 150 may be detached from the outer cover 106 while the end(s) 146, 148 are attached to the outer cover 106 such that the body portion 150 is movable relative to the outer cover 106.

The at least one first fastening element 140 of each rise adjustment feature 102 is provided on and/or coupled to the connecting member 138 such that the at least one first fastening element 140 is connected to the outer cover 106 via the connecting member 138. In the embodiment illustrated, each rise adjustment feature 102 includes three first fastening elements 140; however, the number of first fastening elements 140 provided with rise adjustment feature 102 should not be considered limiting. Moreover, when a plurality of rise adjustment features 102 are utilized, the number of first fastening elements 140 on one rise adjustment feature 102 need not be the same as on another rise adjustment feature 102.

As best illustrated in FIG. 6, the second fastening element 142 of each rise adjustment feature 102 is connected to the outer cover 106. In some embodiments, the second fastening element 142 is connected to the back portion 120, although it need not be in other embodiments (e.g., if the connecting member 138 and at least one first fastening element 140 are provided adjacent to the front portion 118). While a single second fastening element 142 is illustrated with each rise adjustment feature 102, in other embodiments, a plurality of second fastening elements 142 may be provided on the outer cover 106.

The at least one first fastening element 140 and the second fastening element 142 may be various suitable devices or mechanisms that are selectively engageable with each other. As non-limiting examples, the at least one first fastening element 140 and the second fastening element 142 may be hook and loop fasteners, snaps, clips, clasps, adhesive fasteners, cohesive fasteners, mechanical fasteners, male/female mating components, combinations thereof, and/or other suitable features as desired. In the embodiment illustrated, the at least one first fastening element 140 and the second fastening element 142 are male and female snaps. The rise of the diaper 101 is adjusted by engaging a first fastening element 140 with the second fastening element 142. As a non-limiting example, the user may engage the first fastening element 140 furthest from the crotch portion 122 with the second fastening element 142 to provide a first rise adjustment, the user may engage a middle first fastening element 140 with the second fastening element 142 to provide a second rise adjustment different from the first rise adjustment, and the user may engage the first fastening element 140 closest to the crotch portion 122 with the second fastening element 142 to provide a third rise adjustment different from the second rise adjustment. The elastic nature of at least a portion 152 of the strap 144 allows the different first fastening elements 140 to be drawn towards the second fastening element 142, which, in turn, pulls the crotch portion 120 of the chassis 104 upwardly to reduce the rise of the diaper 101. In this way, the rise of the diaper 101 can effectively be “taken in” or “let out” using selective engagement between the first fastening elements 140 and the second fastening element 142. Depending on which of the first fastening elements 140 is engaged with the second fastening element 142, the user may provide various levels of rise adjustment. In other examples, the first fastening elements 140 providing various amounts of rise adjustment may depend on the location of the second fastening element 142 on the outer cover 106. As such, the rise adjustment features 102 may provide better fit and comfort for the wearer 103 while allowing for the overall rise or length of the absorbent article 100 to be adjusted. Additionally, or alternatively, because the rise adjustment features 102 are accessible from an interior of the absorbent article 100, the overall absorbent article 100 may be more durable, such as but not limited to preventing or minimizing inadvertent disengagement from an adjusted rise configuration. As mentioned, the rise adjustment features 102 that are enclosed within the interior of the absorbent article 100 may also prevent and/or minimize contact with the skin of the wearer 103, and such enclosure may further improve durability of the absorbent article 100.

A collection of exemplary embodiments is provided below, including at least some explicitly enumerated as “Embodiments” providing additional description of a variety of example embodiments in accordance with the concepts described herein. These embodiments are not meant to be mutually exclusive, exhaustive, or restrictive; and the disclosure not limited to these example embodiments but rather encompasses all possible modifications and variations within the scope of the issued claims and their equivalents.
Embodiment 1. An absorbent article comprising a rise adjustment feature, the rise adjustment feature comprising: a strap with at least one first fastening element, wherein the strap is configured to be connected to a body-facing side of an outer cover of the absorbent article; and a second fastening element configured to be connected to the outer cover, wherein the at least one first fastening element is configured to engage the second fastening element to adjust a rise of the absorbent article.
Embodiment 2. The absorbent article of any of the preceding or subsequent embodiments or combination of embodiments, wherein the absorbent article is a diaper comprising a front portion, a back portion, and a crotch portion between the front portion and the back portion, and wherein the second fastening element is on the back portion.
Embodiment 3. The absorbent article of any of the preceding or subsequent embodiments or combination of embodiments, wherein the at least one first fastening clement is a snap.
Embodiment 4. The absorbent article of any of the preceding or subsequent embodiments or combination of embodiments, wherein the absorbent article is a reusable diaper.
Embodiment 5. The absorbent article of any of the preceding or subsequent embodiments or combination of embodiments, wherein at least a portion of the strap comprises an elastic material.
Embodiment 6. The absorbent article of any of the preceding or subsequent embodiments or combination of embodiments, further comprising: the outer cover; and a liner material covering at least a portion of the body-facing side of the outer cover, wherein the strap and the at least one first fastening element are positioned between the outer cover and the liner material.
Embodiment 7. The absorbent article of any of the preceding or subsequent embodiments or combination of embodiments, wherein the strap is elongated and comprises opposing ends, and wherein the opposing ends of the strap are connected to the outer cover.
Embodiment 8. An absorbent article comprising a rise adjustment feature, the rise adjustment feature accessible from an interior of the absorbent article, wherein the rise adjustment feature comprises: at least one first fastening element; and a second fastening element, wherein the at least one first fastening element is coupled to a connecting member, and wherein the at least one first fastening element is configured to engage the second fastening element to adjust a rise of the absorbent article.
Embodiment 9. The absorbent article of any of the preceding or subsequent embodiments or combination of embodiments, wherein the elastic material is connected to a body-facing side of an outer cover of the absorbent article.
Embodiment 10. The absorbent article of any of the preceding or subsequent embodiments or combination of embodiments, wherein the absorbent article is a reusable diaper comprising an outer cover comprising a front portion, a back portion, and a crotch portion between the front portion and the back portion, and wherein the second fastening element is on the back portion.
Embodiment 11. The absorbent article of any of the preceding or subsequent embodiments or combination of embodiments, wherein the at least one first fastening element comprises a plurality of first fastening elements coupled to the connecting member.
Embodiment 12. The absorbent article of any of the preceding or subsequent embodiments or combination of embodiments, wherein the connecting member comprises an elastic material.
Embodiment 13. The absorbent article of any of the preceding or subsequent embodiments or combination of embodiments, wherein the connecting member has opposing ends and a body portion between the opposing ends, wherein at least one of the opposing ends is connected to an outer cover of the absorbent article, and wherein the body portion is detached from the outer cover.
Embodiment 14. The absorbent article of any of the preceding or subsequent embodiments or combination of embodiments, further comprising: an outer cover; and a liner material covering at least a portion of a body-facing side of the outer cover, wherein the outer cover and liner material define the interior and wherein the connecting member and the at least one first fastening element are positioned between the outer cover and the liner material.
Embodiment 15. A method of adjusting a rise of an absorbent article, the method comprising: engaging at least one first fastening element of a rise adjustment feature with a second fastening element of the rise adjustment feature, wherein the at least one fastening element is on a strap connected to a body-facing side of an outer cover of the absorbent article, and wherein the second fastening is connected to the outer cover.
Embodiment 16. The method of any of the preceding or subsequent embodiments or combination of embodiments, wherein engaging the at least one first fastening element with the second fastening element comprises accessing the rise adjustment feature from an interior of the absorbent article.
Embodiment 17. The method of any of the preceding or subsequent embodiments or combination of embodiments, wherein engaging the at least one first fastening element with the second fastening element comprises pulling a crotch portion of the absorbent article upwardly to reduce the rise of the absorbent article.
Embodiment 18. The method of any of the preceding or subsequent embodiments or combination of embodiments, wherein the absorbent article is a reusable diaper.
Embodiment 19. The method of any of the preceding or subsequent embodiments or combination of embodiments, wherein the method further comprises adjusting the strap and at least one first fastening element in a location between a liner material and the outer cover.
Embodiment 20. The method of any of the preceding or subsequent embodiments or combination of embodiments, wherein the at least one first fastening element comprises a plurality of first fastening members, and wherein adjusting the rise of the absorbent article comprises engaging one of the plurality of fastening elements with the second fastening element.

The various aspects, embodiments, implementations or features of the described embodiments can be used separately or in any combination. It should be appreciated that the various elements of concepts from the figures may be combined without departing from the spirit or scope of the invention.

The use of the terms “a” and “an” and “the” and similar referents in the context of describing the invention (especially in the context of the following claims) are to be construed to cover both the singular and the plural, unless otherwise indicated herein or clearly contradicted by context. Directional references such as “up,” “down,” “top,” “bottom,” “left,” “right,” “front,” and “back,” among others, are intended to refer to the orientation as illustrated and described in the figure (or figures) to which the components and directions are referencing. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, or gradients thereof, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, the nearness of completion will be so as to have the same overall result as if absolute and total completion were obtained.

Preferred embodiments of this invention are described herein, including the best mode known to the inventors for carrying out the invention. The invention is susceptible to various modifications and alternative constructions, and certain shown exemplary embodiments thereof are shown in the drawings and have been described above in detail. Variations of those preferred embodiments, within the spirit of the present invention, may become apparent to those of ordinary skill in the art upon reading the foregoing description. The inventors expect skilled artisans to employ such variations as appropriate, and the inventors intend for the invention to be practiced otherwise than as specifically described herein. Accordingly, it should be understood that there is no intention to limit the invention to the specific form or forms disclosed, but on the contrary, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of specific embodiments are presented for purposes of illustration and description. They are not intended to be exhaustive or to limit the described embodiments to the precise forms disclosed. It will be apparent to one of ordinary skill in the art that many modifications and variations are possible in view of the above teachings.

That which is claimed:

1. An absorbent article comprising a rise adjustment feature, the rise adjustment feature comprising:
a strap with at least one first fastening element, wherein the strap is configured to be connected to a body-facing side of an outer cover of the absorbent article; and
a second fastening element configured to be connected to the outer cover, wherein the at least one first fastening element is configured to engage the second fastening element to adjust a rise of the absorbent article.

2. The absorbent article of claim 1, wherein the absorbent article is a diaper comprising a front portion, a back portion, and a crotch portion between the front portion and the back portion, and wherein the second fastening element is on the back portion.

3. The absorbent article of claim 1, wherein the at least one first fastening element is a snap.

4. The absorbent article of claim 2, wherein the absorbent article is a reusable diaper.

5. The absorbent article of claim 1, wherein at least a portion of the strap comprises an elastic material.

6. The absorbent article of claim 1, further comprising:
the outer cover; and
a liner material covering at least a portion of the body-facing side of the outer cover,
wherein the strap and the at least one first fastening element are positioned between the outer cover and the liner material.

7. The absorbent article of claim 1, wherein the strap is elongated and comprises opposing ends, and wherein the opposing ends of the strap are connected to the outer cover.

8. An absorbent article comprising a rise adjustment feature, the rise adjustment feature accessible from an interior of the absorbent article, wherein the rise adjustment feature comprises:
at least one first fastening element; and
a second fastening element,
wherein the at least one first fastening element is coupled to a connecting member, and
wherein the at least one first fastening element is configured to engage the second fastening element to adjust a rise of the absorbent article.

9. The absorbent article of claim 8, wherein the elastic material is connected to a body-facing side of an outer cover of the absorbent article.

10. The absorbent article of claim 8, wherein the absorbent article is a reusable diaper comprising an outer cover comprising a front portion, a back portion, and a crotch portion between the front portion and the back portion, and wherein the second fastening element is on the back portion.

11. The absorbent article of claim 8, wherein the at least one first fastening element comprises a plurality of first fastening elements coupled to the connecting member.

12. The absorbent article of claim 8, wherein the connecting member comprises an elastic material.

13. The absorbent article of claim 8, wherein the connecting member has opposing ends and a body portion between the opposing ends, wherein at least one of the opposing ends is connected to an outer cover of the absorbent article, and wherein the body portion is detached from the outer cover.

14. The absorbent article of claim 8, further comprising:
an outer cover; and
a liner material covering at least a portion of a body-facing side of the outer cover,
wherein the outer cover and liner material define the interior and wherein the connecting member and the at least one first fastening element are positioned between the outer cover and the liner material.

15. A method of adjusting a rise of an absorbent article, the method comprising:
engaging at least one first fastening element of a rise adjustment feature with a second fastening element of the rise adjustment feature,
wherein the at least one fastening element is on a strap connected to a body-facing side of an outer cover of the absorbent article, and
wherein the second fastening is connected to the outer cover.

16. The method of claim 15, wherein engaging the at least one first fastening element with the second fastening element comprises accessing the rise adjustment feature from an interior of the absorbent article.

17. The method of claim 15, wherein engaging the at least one first fastening element with the second fastening element comprises pulling a crotch portion of the absorbent article upwardly to reduce the rise of the absorbent article.

18. The method of claim 15, wherein the absorbent article is a reusable diaper.

19. The method of claim 18, wherein the method further comprises adjusting the strap and at least one first fastening element in a location between a liner material and the outer cover.

20. The method of claim 15, wherein the at least one first fastening element comprises a plurality of first fastening members, and wherein adjusting the rise of the absorbent article comprises engaging one of the plurality of fastening elements with the second fastening element.
